(19) United States
(12) Patent Application Publication
(10) Pub. No.: US 2026/0340214 A1
(43) Pub. Date: Oct. 29, 2026

(54) INVERSE DESIGN OF OBJECTS WITH A RELEASE PROFILE

(71) Applicants: Max-Planck-Gesellschaft zur Förderung der Wissenschaften e.V., München (DE); THE REGENTS OF THE UNIVERSITY OF CALIFORNIA, Oakland, CA (US)

(72) Inventors: Francis Julian PANETTA, Davis, CA (US); Vahid BABAEI, München (DE); Halehossadat Mohammadian ESFAHANI, München (DE); Emiliano LUCI, München (DE); Hans-Peter SEIDEL, München (DE)

(73) Assignees: Max-Planck-Gesellschaft zur Förderung der Wissenschaften e.V., München (DE); THE REGENTS OF THE UNIVERSITY OF CALIFORNIA, Oakland, CA (US)

(21) Appl. No.: 19/117,562

(22) PCT Filed: Sep. 29, 2023
(86) PCT No.: PCT/EP2023/077032

§ 371 Date: Apr. 1, 2025

Related U.S. Application Data
(60) Provisional application No. 63/413,016, filed on Oct. 4, 2022.

(51) Int. Cl. G06F 30/17

(52) U.S. Cl. CPC G06F 30/17

(57) ABSTRACT

Disclosed herein is a manufacturing system (100, 300). The execution of the machine executable instructions (112) causes a computational system (104) to: receive (200) a release profile (114, 720, 722) for an object to be constructed of one or more materials; construct (202) a similarity cost function (116) dependent on a difference between a simulated release curve (118) of a trial density field (120) and the release profile for the object; construct (204) an optimization problem (122) comprising the similarity cost function and a manufacturing penalty function (124); construct (206) an optimized density field (126) by numerically solving the optimization problem to provide the trial density field that minimizes the optimization problem; and provide (210) a three-dimensional design (130, 730, 732, 734) of the object using the optimized density field that minimizes the optimization problem.

### FIELD OF THE INVENTION

The invention relates to design of objects to have a release profile.

### BACKGROUND AND RELATED ART

Many objects, such as pharmaceuticals, are designed to release one or more materials as a function of time. Often times it is desired to release the one or more materials at a particular rate. The release profile of an object for a material as used herein refers to the rate at which the material is released into a predetermined solvent. A cumulative release profile is the total, cumulative amount of material released as a function of time. The instantaneous release profile is the derivative of the cumulative release profile and is a measure of how much of the material the object is currently releasing as a function of time. References to a release profile are understood to refer to both of these as specifying one of the cumulative release profile and the instantaneous release profile is mathematically equivalent to specifying the other.

### SUMMARY

The invention provides for a manufacturing system, a computer program, and a method in the independent claims. Embodiments are given in the dependent claims.

The design of objects which are able to release a controlled amount of one or more materials into a predetermined solvent is useful in a variety of technical fields. A difficulty is that it can be difficult to design an object so that it has a particular release profile. Designing an object with a release profile may involve a large amount of trial and error. Embodiments may provide for an improved means of providing a three-dimensional design of an object with a release profile. This may be achieved by solving an optimization problem for an optimized density field which is then translated into the three-dimensional design. The optimization problem comprises a similarity cost function and a manufacturing penalty function. The manufacturing penalty function can be used to penalize trial density fields which do not satisfy a manufacturing constraint for a fabrication machine. For example, if the object is to be three-dimensionally printed one could add manufacturing constraints that would produce a design which could be printed by a particular three-dimensional printer. The similarity cost function provides a measure between the release profile that is being designed and for a simulated release function of a trial density field.

In one aspect the invention provides for a manufacturing system that comprises a memory storing machine-executable instructions. The manufacturing system further comprises a computational system. Execution of the machine-executable instructions causes the computational system to receive a release profile for an object to be constructed of one or more materials. The release profile is descriptive of a dissolution of the one or more materials in a predetermined solvent. At this point the object has an arbitrary shape is has not yet been designed. Execution of the machine-executable instructions further causes the computational system to construct a similarity cost function dependent on a difference between a simulated release curve of a trial density field and the release profile for the object. The terms “release curve” and “release profile” are equivalent and are used interchangeably.

The trial density field is a density field descriptive of the density of the one or more materials three-dimensionally. The trial density field is essentially a density field in the similarity cost function used for the optimization and design.

Execution of the machine-executable instructions further causes the computational system to construct an optimization problem comprising the similarity cost function and a manufacturing penalty function. The manufacturing penalty function comprises one or more manufacturing constraints for a fabrication machine. For example, the manufacturing penalty function could be used to specify or control how the optimization problem fits for a particular manufacturing modality provided by the fabrication machine.

Examples of manufacturing constraints may include, but are not limited to: a density constraint of the one or more materials, a spatially dependent density constraint of the one or more materials, a layer thickness constraint, a surface roughness constraint, a binary density constraint, a self-supporting layer constraint, a cutting tool profile constraint, a mold removability constraint, and a material continuity constraint, and combinations thereof.

Execution of the machine-executable instructions further causes the computational system to construct an optimized density field by numerically solving the optimization problem to provide the trial density field that minimizes the optimization problem. The optimized density field is then the density field which minimizes the difference between the trial density field and the release profile for the object to be designed.

Execution of the machine-executable instructions further causes the computational system to provide a three-dimensional design of the object using the optimized density field that minimizes the optimization problem. In this step the optimized density field is put into a form which may be used for the manufacturing by the fabrication machine. For example, if the three-dimensional design were to be three-dimensional printed the three-dimensional design could be a conversion of the optimized density field into an STL file or other type of file, which may be used for controlling or generating machine commands for controlling the fabrication machine.

In another embodiment the density of each of the one or more materials varies spatially. This may have different means in different examples. For example, if there were a single material then this could include a change in the density for a foam, possibly by causing voids, a porous microstructure or other holes within the material. In other examples, the components of at a particular location may be mixed. If there are multiple materials then this may include halftoning of the design and specifying that each voxel is filled with a particular material. For example, with three-dimensional printing this may include depositing each voxel with different three-dimensional printed materials.

In another embodiment the simulated release curve of the trial density field is formulated as an integral of a pointwise release curve of the trial density field. This embodiment may be beneficial because it may provide for an efficient means of solving the optimization problem numerically. The pointwise release curve may also be averaged over predetermined volumes. For example, the pointwise release curve of the trial density field may be voxelized such that there is a spatial averaging effect in the calculation.

In another embodiment the pointwise release curve of the trial density field is determined for each point within the trial density field by determining a distance from each point to a closest boundary of the trial density field and assuming a constant dissolution rate of each of the one or more materials in the predetermined solvent. This is, for example, a constant rate that is dependent upon which material. For example, one material may have a different dissolution rate than another. This embodiment may be beneficial because it provides for an extremely numerically efficient means of calculating the pointwise release curve.

In another embodiment the distance to each point within the trial density field to the closest boundary of the trial density field is determined using a fast marching algorithm or a geodesics in heat algorithm. If the trial density field has been discretized into voxels then this operation is performed for each voxel within the trial density field. These algorithms may be beneficial because they provide an efficient means of solving the distance for each point within the trial density field in a numerically efficient manner. These equations may be solved continuously. Spatial averaging may be used to accelerate the problem numerically. For example, a tetrahedral mesh or voxels may be used to choose how large the spatial averaging is.

In another embodiment the trial density field is discretized into voxels.

This embodiment may be beneficial because it may provide for a means of accelerating the numerical algorithm.

In another embodiment the voxels contribute to the simulated release curve using a scaled linear ramp function. In other words, the voxels do not dissolve immediately but dissolve linearly overtime. This may be beneficial because it is numerically efficient to calculate and may provide an accurate simulation of the release curve.

In another embodiment the optimization problem comprises a density projection filter. The use of a trial density field may provide for an improved means of estimating the optimal density field using a discretized approximation.

In another embodiment the optimization problem comprises a smoothing filter. For example, the smoothing filter may be used between iterations. The use of a smoothing filter may contribute to the optimization problem by causing a more rapid convergence to the optimized density field. The use of a smoothing filter may also increase the manufacturing by limiting, for example, the surface roughness of the object specified by the optimized density field.

In another embodiment the smoothing filter linearly decays over a predetermined smoothing radius. This embodiment may be beneficial because it may serve as a means of controlling the optimization problem and controlling the feature size and smoothness.

In another embodiment the optimization problem is formulated using a two-dimensional blueprint density field. In this example, the two-dimensional blueprint density field is used to provide a two-dimensional shape which is then expanded to a three-dimensional shape, for example, for providing an extruded shape. This may provide for an improved means of accelerating the optimization problem, and it may also provide for a means of providing for a more manufacturable three-dimensional design. For example, it may enable inexpensive manufacturing techniques such as using an extruder or mold.

In another embodiment the computational system is configured to transform the two-dimensional blueprint density field into an extruded three-dimensional density field to provide the trial density field. This embodiment may be beneficial because it provides for a design which may be easily manufactured by an extruder or mold. It may also be advantageous because it may provide for a shape which may be three-dimensional printed in which the support of various layers does not need to be considered.

In another embodiment the optimization problem is solved using a gradient-based optimization algorithm. This may be beneficial because gradient-based methods converge to a solution rapidly.

In another embodiment the gradient-based optimization algorithm is an interior-point Limited-memory Broyden-Fletcher-Goldfarb-Shanno (L-BFGS) algorithm. This embodiment may be beneficial because it provides for a very efficient means of providing a gradient-based optimization algorithm.

In another embodiment the optimization problem is solved using a method of moving asymptotes algorithm.

In another embodiment the optimization problem is solved using a genetic algorithm. For example, the trial density field may be discretized and then may be modified several times during each iteration. The best design may be taken to be iterated or there may be, for example, a blending between the several best solutions to produce a number of trial density fields for the next iteration. In any case, the genetic algorithm may be implemented in a straightforward and easy way. The numerical method may be easier to implement.

In another embodiment the optimization problem is solved using a simulated annealing method algorithm.

In another embodiment providing the three-dimensional design of the object using the optimized density field that minimizes the optimization problem comprises applying a post-processing algorithm to the optimized density field. Instead of providing the optimized density field directly, a filter or other algorithm may be used to take the optimized density field and then provide the three-dimensional design.

In another embodiment the post-processing algorithm is a halftoning algorithm.

In another embodiment the post-processing algorithm is a microstructure approximation algorithm. The microstructure approximation algorithm may for example, provide the three-dimensional design with various voids or holes within it. This may be a means of designing a foam or foamlike structure with a chosen density that varies spatially.

In another embodiment the post-processing algorithm may be a surface contouring algorithm. This may be beneficial because it may provide for a means of providing an optimal surface of the three-dimensional design for a particular manufacturing modality.

In another embodiment the release profile is a zero-order release curve. This embodiment may be particularly beneficial because by other means it may not be possible to design an object which dissolves and provides a zero-order release curve. In this embodiment the one or more materials is preferably a single-material.

In another embodiment the one or more materials is a single material.

In another embodiment the one or more materials is multiple materials.

In another embodiment at least one or more materials is or comprises a foam.

In another embodiment the foam is designed using holes or voids. This may, for example, be an excellent way of using a solid material and then custom designing the density of the foam at a particular location.

In another embodiment the at least one or more materials comprises a pharmaceutical.

In another embodiment the at least one or more materials comprises a catalyst.

In another embodiment the at least one or more materials comprises an enzyme.

In another embodiment the at least one or more materials comprises a flavoring.

In another embodiment the at least one or more materials comprises a perfume.

In another embodiment the at least one or more materials comprises a deodorant.

In another embodiment the at least one or more materials comprises a pigment.

In another embodiment the at least one or more materials comprises an antibacterial agent.

In another embodiment the at least one or more materials comprises a bio ink, particularly in three-dimensional printing processes.

In another embodiment the at least one or more materials comprises a collagen. The use of collagen may be beneficial because this may be useful for example, in three-dimensional printing or designing structures which can be used to provide a scaffolding for bone tissue.

In another embodiment the at least one or more materials comprises a hydro gel.

In another embodiment execution of the machine-executable instructions further causes the computational system to generate fabrication machine control commands using the three-dimensional design. The fabrication machine control commands are configured for controlling the fabrication machine. This embodiment may be beneficial because instead of providing the three-dimensional design alone, the three-dimensional design has been used to construct the codes for controlling a particular fabrication machine for manufacturing the object or for controlling the machine for designing a tool for manufacturing the object.

In another embodiment the manufacturing system further comprises the fabrication machine. Execution of the machine-executable instructions further causes the fabrication machine to execute the fabrication machine commands. This may, for example, be in some instances, for manufacturing the object or for performing a step in manufacturing the object such as providing for tooling, molds or other materials used for manufacturing.

In another embodiment the fabrication machine is an additive fabrication machine. Execution of the fabrication machine commands causes the fabrication machine to manufacture the object. This embodiment may be beneficial because it causes the fabrication machine to manufacture the object correctly.

In another embodiment the fabrication machine is a stereolithography three-dimensional printer.

In another embodiment the fabrication machine is a digital light processing three-dimensional printer.

In another embodiment the fabrication machine is an extrusion three-dimensional printer.

In another embodiment the fabrication machine is an inkjet printer.

In another embodiment the fabrication machine is a powder-bed inkjet three-dimensional printer.

In another embodiment the manufacturing penalty function comprises a density constraint of the one or more materials.

In another embodiment the penalty function comprises a spatially dependent density constraint of the one or more materials.

In another embodiment the penalty function further comprises a layer thickness constraint. This, for example, may be particularly useful in extruded or three-dimensional printed manufacturing processes.

In another embodiment the manufacturing function further comprises a surface roughness constraint. Certain manufacturing processes such as three-dimensional printing may have a limit on how smooth a surface can be.

In another embodiment the manufacturing penalty function further comprises a binary density constraint. This may be particularly useful in enforcing the optimization problem to provide a correct solution for a solid object.

In another embodiment the manufacturing penalty function further comprises a self-supporting layer constraint. This, for example, may be useful in three-dimensional printing.

In another embodiment the fabrication machine is configured to fabricate a tool for manufacturing an object. Execution of the fabrication machine commands causes the fabrication machine to manufacture the tool. This may, for example, be useful in mass production situations where the fabrication machine commands are not used to manufacture the object correctly but are used to prepare a manufacturing system for manufacturing the object.

In another embodiment the tool is a mold.

In another embodiment the tool is an injection mold.

In another embodiment the tool is a pill press.

In another embodiment the fabrication machine is a milling machine.

In another embodiment the fabrication machine is a subtractive manufacturing machine.

In another embodiment the fabrication machine is a three-dimensional printer.

In another embodiment the fabrication machine is a metal three-dimensional printer.

In another embodiment the fabrication machine is a directed energy deposition printer.

In another embodiment the fabrication machine is a powder bed fusion printer.

In another embodiment the manufacturing penalty function comprises a surface roughness constraint. This may, for example, be useful in limiting the three-dimensional design to a design that may be manufactured.

In another embodiment the manufacturing penalty function comprises a cutting tool profile constraint. For example, if the fabrication machine is a milling machine there may be a limit to the shapes which can be milled.

In another embodiment the manufacturing penalty function comprises a mold removability constraint. For example, if the fabrication machine is an injection molding system the design may be designed such that the object can be removed once it has been manufactured from a mold.

In another embodiment the manufacturing penalty function comprises a continuous material restraint. For example, if the object is manufactured using a mold or a press it may require the object to be continuous throughout.

In another aspect the invention provides for a computer program that comprises machine-executable instructions for execution by a computational system. The computer program may, for example, be stored on a non-transitory storage medium. Execution of the machine-executable instructions causes the computational system to receive a release profile for an object to be constructed of one or more materials. The release profile is descriptive of the dissolution of the one or more materials in a predetermined solvent. Execution of the machine-executable instructions further causes the computational system to construct a similarity cost function that is dependent on a difference between a simulated release curve of a trial density field and the release profile for the object. Execution of the machine-executable instructions further causes the computational system to construct an optimization problem comprising the similarity cost function and a manufacturing penalty function. The manufacturing penalty function comprises one or more manufacturing constraints for a fabrication machine. Execution of the machine-executable instructions further causes the computational system to construct an optimized density field by numerically solving the optimization problem to provide the trial density field that minimizes the optimization problem. Execution of the machine-executable instructions further causes the computational system to provide a three-dimensional design of the object using the optimized density field that minimizes the optimization problem.

In another aspect the method provides for a method. The method may, for example, be a method of providing a three-dimensional design of an object with a particular release profile; it may also be a method of manufacturing the object or manufacturing a tool for producing the object.

The method comprises receiving a release profile for an object to be constructed of one or more materials. The release profile is descriptive of the dissolution of the one or more materials in a predetermined solvent. The method further comprises constructing a similarity cost function that is dependent on the difference between a simulated release curve of a trial density field and the release profile for the object. The method further comprises constructing an optimization problem comprising the similarity cost function and a manufacturing penalty function. The manufacturing penalty function comprises one or more manufacturing constraints for a fabrication machine.

The method further comprises constructing an optimized density field by numerically solving the optimization problem to provide the trial density field that minimizes the optimization problem. The method further comprises providing a three-dimensional design of the object using the optimized density field that minimizes the optimization problem.

The method further comprises manufacturing the object using the three-dimensional design. This may entail manufacturing the object directly with the fabrication machine or using the fabrication machine to make a tool or fixtures for manufacturing the object.

In another embodiment the release curve profile is a zero-order release curve.

In another embodiment the method further comprises receiving a set of three-dimensional designs for multiple calibration objects. The method further comprises manufacturing the multiple calibration objects using the set of three-dimensional designs. Again, this may be either manufacturing the multiple calibration objects directly using the fabrication machine or it may involve manufacturing a tool for a manufacturing system for producing the multiple calibration objects.

The method further comprises calculating a simulated release curve for each of the multiple calibration objects. For example, this may be done in the same way as was done during construction of the optimization problem.

The method further comprises repeatedly measuring a concentration of the one or more materials in the predetermined solvent as a function of time as each of the multiple calibration objects dissolve in the predetermined solvent. The method further comprises constructing a measured release profile for each of the multiple calibrations object using the concentration of the one or more materials in the predetermined solvent as a function of time. Then the method further comprises performing a calibration of the optimization problem by calculating a fitting between the measured release profile and the simulated release profile of each of the multiple calibration objects. This embodiment may be beneficial particularly when performed before providing the three-dimensional design. This may, for example, be useful in making release profile of the three-dimensional design more accurate.

In some examples, the measurements and simulations of dissolution may have different time and concentration units. The above-described calibration may be useful in this situation. A measurement may, for example, be done in physical units (g/kg and seconds), a simulation may possibly use non-physical or normalized units for both time and concentration. Calibrating the system, such as the above calibration, may comprise building a mapping between these units. For this purpose, one may print or fabricate a set of different calibration objects of equal mass, measure their release profiles, and simulate them using a forward model. One may assume that there exist two decoupled linear mappings between the physical concentration and time units and their simulation counterparts. The scalar coefficient defining the linear map for concentration is given simply by the ratio of the final solution concentration in physical units (determined precisely by weighing the object before dissolution) to the simulated final concentration \tilde{C}(T). The time mapping is computed by minimizing the following cost function for the scalar coefficient k∈R.

$$ℒ ( {C}_{s} , {C}_{r} , k ) = \int {w}_{s} \vert {kC}_{s}^{′} ( kt ) - {C}_{r}^{′} ( t ) \vert + \vert {C}_{s} ( kt ) - {C}_{r} ( t ) \vert dt ,$$

where Cₛ and Cᵣ are the cumulative release profiles in simulation time and in real time, respectively, and weight parameter wₛ (set to 1 in this example) adjusts the relative importance of fitting the curve slopes.

The calibration step may also be used to compensate for mismatches between digital designs and their fabricated counterparts. While some examples may assume a homogeneously filled, isotropic solid, the parts fabricated using FDM exhibit prominent infill-dependent heterogeneities and highly anisotropic properties aligned with the filament direction.

In another aspect the invention provides for an object manufactured according to an embodiment of the method. This, for example, may provide for manufactured objects that have tailored release profiles. In particular it may enable the production of a release profile for the object that is a zero-order release curve.

It is understood that one or more of the aforementioned embodiments of the invention may be combined as long as the combined embodiments are not mutually exclusive.

As will be appreciated by one skilled in the art, aspects of the present invention may be embodied as an apparatus, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer executable code embodied thereon.

Any combination of one or more computer readable medium(s) may be utilized. The computer readable medium may be a computer readable signal medium or a computer readable storage medium. A ‘computer-readable storage medium’ as used herein encompasses any tangible storage medium which may store instructions which are executable by a processor or computational system of a computing device. The computer-readable storage medium may be referred to as a computer-readable non-transitory storage medium. The computer-readable storage medium may also be referred to as a tangible computer readable medium. In some embodiments, a computer-readable storage medium may also be able to store data which is able to be accessed by the computational system of the computing device. Examples of computer-readable storage media include, but are not limited to: a floppy disk, a magnetic hard disk drive, a solid-state hard disk, flash memory, a USB thumb drive, Random Access Memory (RAM), Read Only Memory (ROM), an optical disk, a magneto-optical disk, and the register file of the computational system. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example, data may be retrieved over a modem, over the internet, or over a local area network. Computer executable code embodied on a computer readable medium may be transmitted using any appropriate medium, including but not limited to wireless, wire line, optical fiber cable, RF, etc., or any suitable combination of the foregoing.

A computer readable signal medium may include a propagated data signal with computer executable code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

‘Computer memory’ or ‘memory’ is an example of a computer-readable storage medium. Computer memory is any memory which is directly accessible to a computational system. ‘Computer storage’ or ‘storage’ is a further example of a computer-readable storage medium. Computer storage is any non-volatile computer-readable storage medium. In some embodiments computer storage may also be computer memory or vice versa.

A ‘computational system’ as used herein encompasses an electronic component which is able to execute a program or machine executable instruction or computer executable code. References to the computational system comprising the example of “a computational system” should be interpreted as possibly containing more than one computational system or processing core. The computational system may for instance be a multi-core processor. A computational system may also refer to a collection of computational systems within a single computer system or distributed amongst multiple computer systems. The term computational system should also be interpreted to possibly refer to a collection or network of computing devices each comprising a processor or computational systems. The machine executable code or instructions may be executed by multiple computational systems or processors that may be within the same computing device or which may even be distributed across multiple computing devices.

Machine executable instructions or computer executable code may comprise instructions or a program which causes a processor or other computational system to perform an aspect of the present invention. Computer executable code for carrying out operations for aspects of the present invention may be written in any combination of one or more programming languages, including an object-oriented programming language such as Java, Smalltalk, C++ or the like and conventional procedural programming languages, such as the “C” programming language or similar programming languages and compiled into machine executable instructions. In some instances, the computer executable code may be in the form of a high-level language or in a pre-compiled form and be used in conjunction with an interpreter which generates the machine executable instructions on the fly. In other instances, the machine executable instructions or computer executable code may be in the form of programming for programmable logic gate arrays.

The computer executable code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described with reference to flowchart illustrations and/or block diagrams of methods, apparatus (systems) and computer program products according to embodiments of the invention. It is understood that each block or a portion of the blocks of the flowchart, illustrations, and/or block diagrams, can be implemented by computer program instructions in form of computer executable code when applicable. It is further under stood that, when not mutually exclusive, combinations of blocks in different flowcharts, illustrations, and/or block diagrams may be combined. These computer program instructions may be provided to a computational system of a general-purpose computer, special purpose computer, or other programmable data processing apparatus to produce a machine, such that the instructions, which execute via the computational system of the computer or other programmable data processing apparatus, create means for implementing the functions/acts specified in the flowchart and/or block diagram block or blocks.

These machine executable instructions or computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The machine executable instructions or computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

A ‘user interface’ as used herein is an interface which allows a user or operator to interact with a computer or computer system. A ‘user interface’ may also be referred to as a ‘human interface device.’ A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer to indicate the effects of the operator's control or manipulation. The display of data or information on a display or a graphical user interface is an example of providing information to an operator. The receiving of data through a keyboard, mouse, trackball, touchpad, pointing stick, graphics tablet, joystick, gamepad, webcam, headset, pedals, wired glove, remote control, and accelerometer are all examples of user interface components which enable the receiving of information or data from an operator.

A ‘hardware interface’ as used herein encompasses an interface which enables the computational system of a computer system to interact with and/or control an external computing device and/or apparatus. A hardware interface may allow a computational system to send control signals or instructions to an external computing device and/or apparatus. A hardware interface may also enable a computational system to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A ‘display’ or ‘display device’ as used herein encompasses an output device or a user interface adapted for displaying images or data. A display may output visual, audio, and or tactile data. Examples of a display include, but are not limited to: a computer monitor, a television screen, a touch screen, tactile electronic display, Braille screen, Cathode ray tube (CRT), Storage tube, Bi-stable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, and Head-mounted display.

### BRIEF DESCRIPTION OF THE DRAWINGS

In the following embodiments of the invention are explained in greater detail, by way of example only, making reference to the drawings in which:

FIG. 1 illustrates an example of a manufacturing system;

FIG. 2 shows a flow chart which illustrates a method of operating the manufacturing system of FIG. 1;

FIG. 3 illustrates a further example of a manufacturing system;

FIG. 4 shows a flow chart which illustrates a method of operating the manufacturing system of FIG. 3;

FIG. 5 illustrates an example of a trial density field and its dissolution time field;

FIG. 6 illustrates both a graph of a voxel release function for three voxels and a release profile for an object containing these voxels; and

FIG. 7 illustrates several three-dimensional designs.

### DETAILED DESCRIPTION

Like numbered elements in these figures are either equivalent elements or perform the same function. Elements which have been discussed previously will not necessarily be discussed in later figures if the function is equivalent.

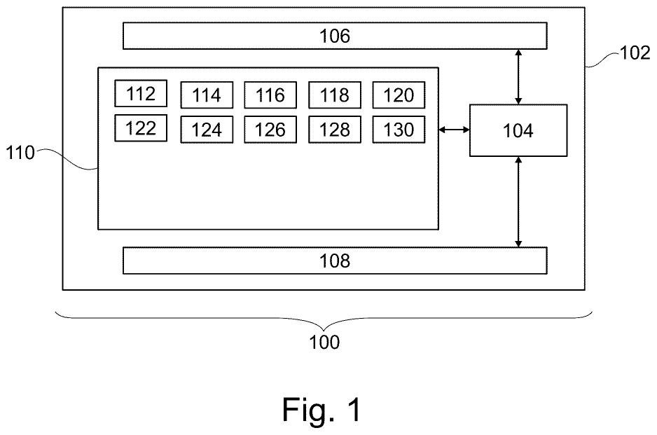

FIG. 1 illustrates an example of a manufacturing system 100. In this example the manufacturing system comprises a computer 102. The computer 102 may represent one or more computing systems at one or more locations. The computer 102 could, for example, be part of a controller for a fabrication machine; it could also be a system implementing a CAD or design program that is configured for assisting someone in providing for making three-dimensional designs.

The computer 102 is shown as comprising a computational system 104. Likewise, the computational system 104 may represent one or more computational systems or cores that are located at one or more locations. In this example, the computational system 104 is shown as being connected with an optional hardware 106 and an optional user interface 108. The hardware interface 106, if it is present, may enable the computational system 104 to communicate with and control other components of the manufacturing system such as a fabrication machine. The user interface 108 may, for example, be used by an operator or controller to control the operation and function of the manufacturing system.

The memory 110 is intended to represent various types of memory which are accessible to the computational system 104. In some examples the memory 110 may be a non-transitory storage medium. The memory 110 is shown as containing machine-executable instructions 112. The machine-executable instructions 112 enable the computational system 104 to perform such things as numerical computations and the control of other components. For example, the various numerical steps and algorithms may be implemented in the machine-executable instructions 112.

The memory 110 is further shown as containing a release profile for an object that is to be constructed of one or more materials. In essence, this release profile may be a desired release profile and the manufacturing system may be configured for providing a three-dimensional design of an object which satisfies this release profile or even, in some instances, manufactures it. The memory 110 is further shown as containing a similarity cost function 116. The similarity cost function is dependent on a difference between a simulated release curve 118 of a trial density field 120 and the release profile 114. The memory 110 is shown as further containing an optimization problem 122 comprising the similarity cost function 116 and a manufacturing penalty function 124.

The manufacturing penalty function 124 ensures that certain constraints are forced on the optimization problem to provide for certain aspects of manufacturability. The manufacturing penalty function comprises one or more manufacturing constraints for a fabrication machine. For example, the manufacturing penalty function may blow up or have a large value if the trial density field has a shape or density which is not able to be manufactured by the fabrication machine.

The memory 110 is further shown as containing an optimized density field 126 that was provided by numerically solving the optimization problem 122 to provide a trial density field that minimizes the optimization problem 122. The memory 110 is further shown as containing an optional post-processing algorithm 128 that may be applied to the optimized density field 126. This, for example, may provide such things as forcing a surface roughness constraint, a cutting tool profile, a mold removability constraint or enforcing certain continuous aspects of the design. The memory 110 is further shown as containing the three-dimensional design which is a design for the object that has the release profile 114.

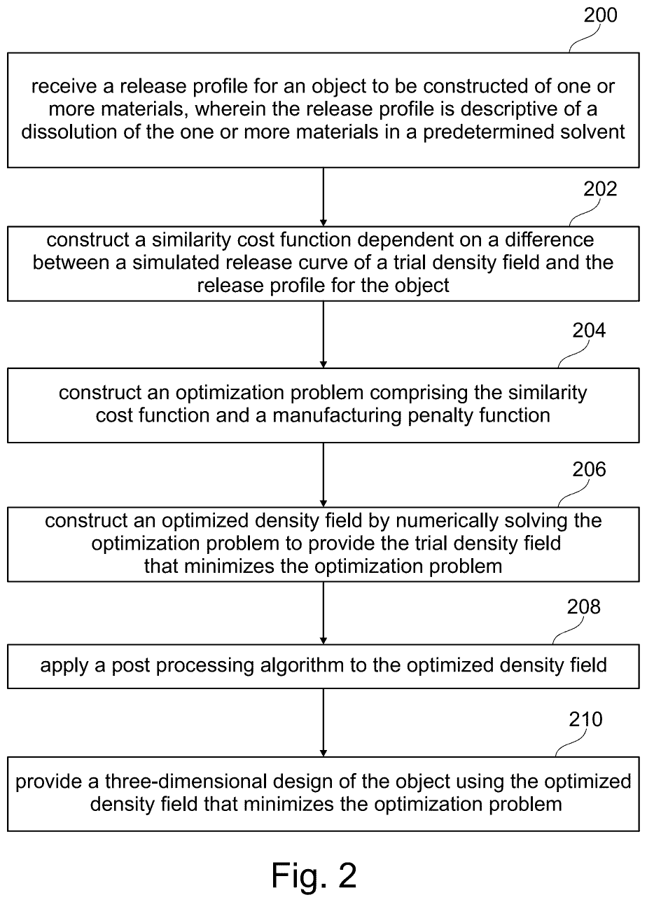

FIG. 2 shows a flowchart which illustrates a method of operating the manufacturing system 100 of FIG. 1. First, in step 200, the release profile 114 is received for an object that is to be constructed of one or more materials. The release profile is descriptive of a dissolution of the one or more materials in a predetermined solvent. Next, in step 202, the similarity cost function 116 is constructed. The similarity cost function 116 is dependent on a difference between a simulated release curve 118 of the trial density function 120 and the release profile 114 for the object. Then, in step 204, the optimization problem 122 is constructed.

The optimization problem 122 comprises the similarity cost function 116 and a manufacturing penalty function 124. The manufacturing penalty function comprises one or more manufacturing constraints for a fabrication machine. Next, in step 206, an optimized density field 126 is constructed by numerically solving the optimization problem 122 to provide the trial density field 120 that minimizes the optimization problem 122. Next, step 208 is optionally performed. In step 208 a post-processing algorithm 128 is applied to the optimized density field 126. And finally, in step 210, the three-dimensional design 130 is provided using the optimized density field 126. In some cases, this may entail using the optimized density field 126 after it has been processed by the post-processing algorithm 128.

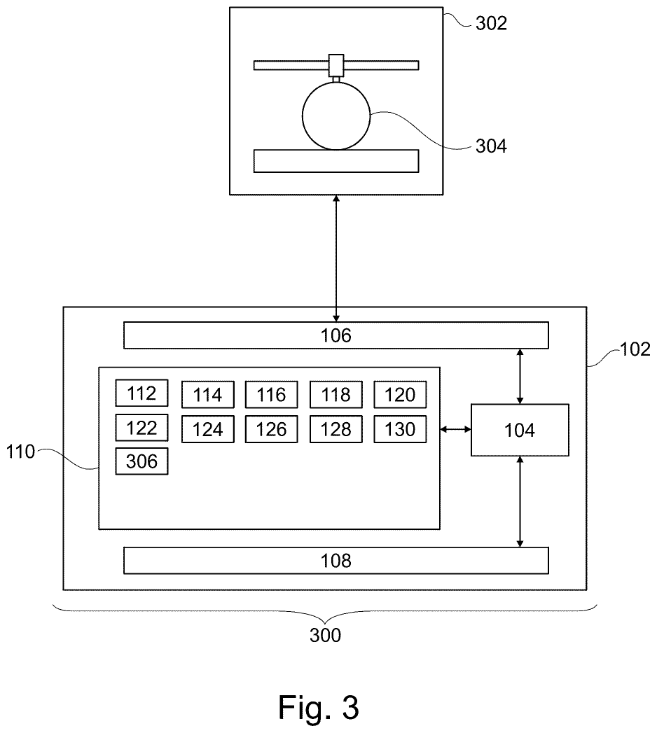

FIG. 3 illustrates a further example of a manufacturing system 300. The example 300 illustrated in FIG. 3 is similar to the example 100 illustrated in FIG. 1 except it additionally comprises a fabrication machine 302. The fabrication machine 302 can be seen as being in the process of manufacturing an object or tool 304.

The memory 110 is further shown as containing fabrication machine control commands 306 that have been constructed using the three-dimensional design 130. The fabrication machine control commands 306 are able to be executed by the fabrication machine 302 and as a result, causes the fabrication machine 302 to produce the object or tool 304.

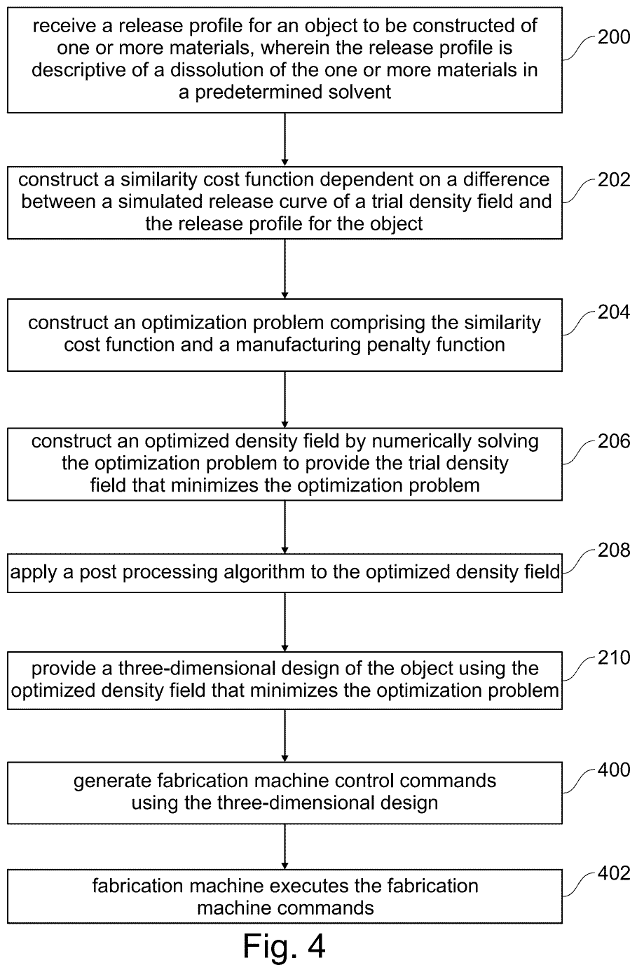

FIG. 4 shows a flowchart which illustrates a method of operating the manufacturing system 300 of FIG. 3. The method in FIG. 4 is similar to the method in FIG. 2. The method illustrated in FIG. 4 begins with performing steps 200-210 as was illustrated in FIG. 1. After step 210 has been performed, step 400 is performed. In step 400, the computational system 104 generates the fabrication machine control commands 306 using the three-dimensional design 130. Next, in step 402, the computational system 104 causes the fabrication machine 302 to execute the fabrication control commands 306 and thereby causes it to manufacture the object or tool 304.

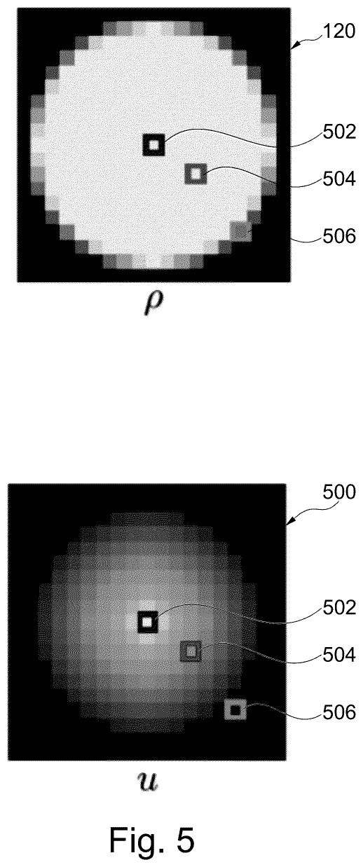

FIG. 5 illustrates an example of a trial density field 120 and its dissolution time field 500. In this example, the equations have been made discrete. Voxels 502, 504, and 506 are indicated in both images 120 and 500.

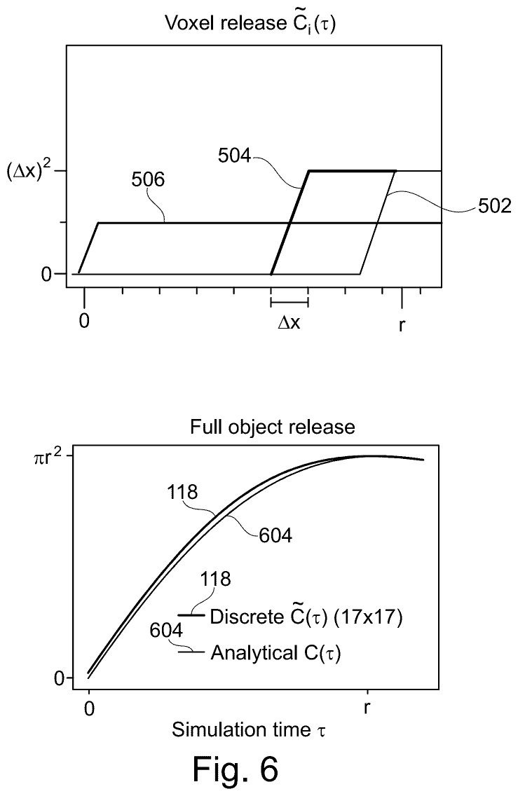

FIG. 6 illustrates a graph of the voxel release function for the voxels 502, 504, and 506. It can be seen that the voxel 502 at the center of the object releases much later than the other two voxels. Below this graph, an additional graph for the full object release profile 602 is illustrated. There, the difference between the simulated release curve 118 and an analytically calculated release curve 604 is shown. It can be seen that the agreement between the two curves 118 and 604 is quite good.

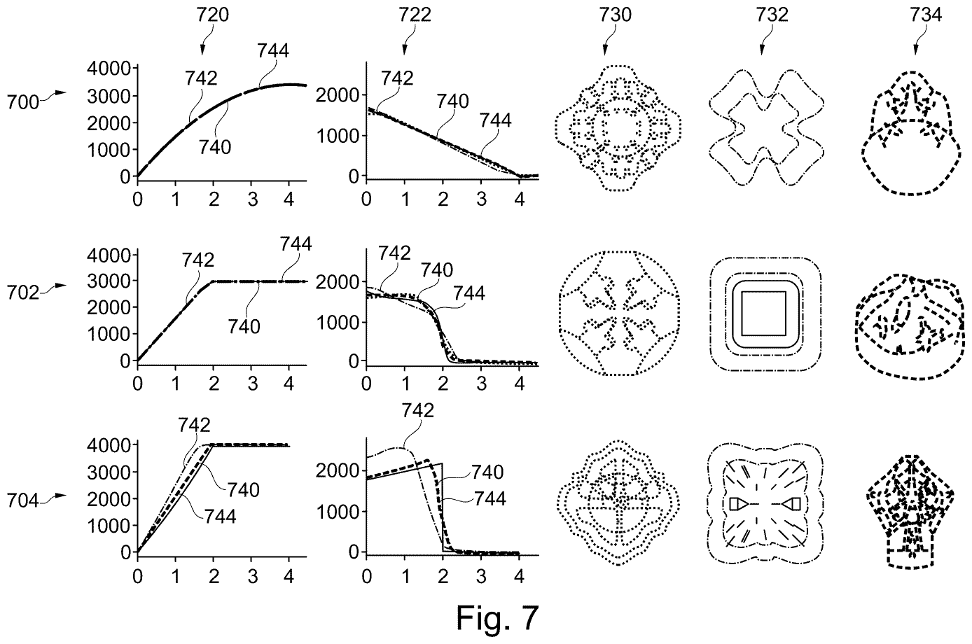

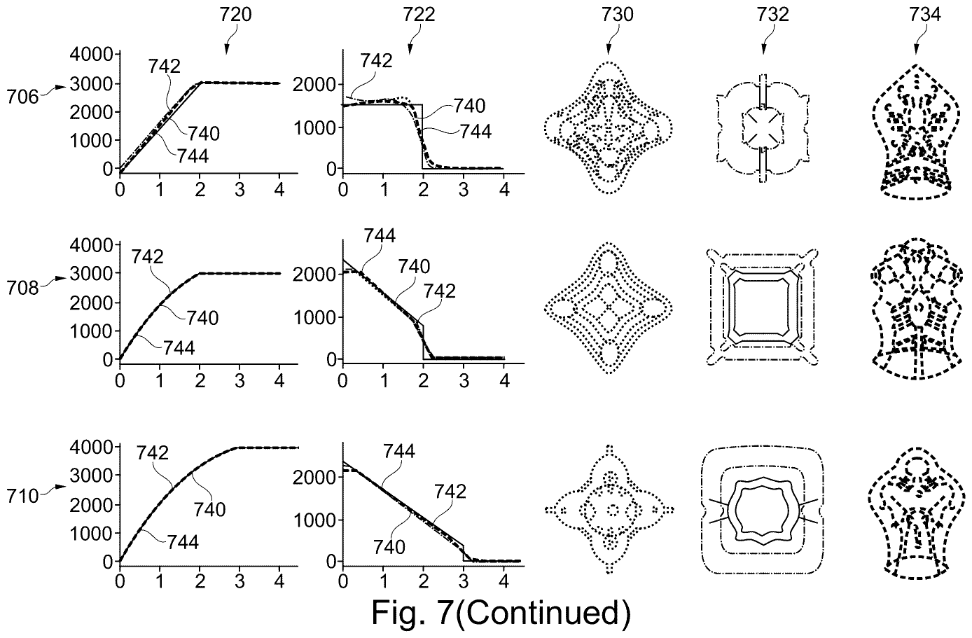

FIG. 7 illustrates a number of designs resulting from an example. The designs are classified according to a constraint provided by a cumulative release curve 720 and its equivalent instantaneous release curve. The different design constraints are shown in rows. There are individual rows for design constraint 700, design constraint 702, design constraint 704, design constraint 706, design constraint 708, design constraint 710.

For each of these three design constraints 700, 702, 704, 706, 708, 710 three designs were produced using additional constraints. Objects in Column 730 have a minimal fabricability constraint. Objects in column 730 are continuous, however further constraints were not imposed. Objects in column 732 are formed from the extrusion of a two-dimensional density field and can be used to design extruded objects. Objects in column 734 have an incremental compliance (ICO) constraint in the manufacturing penalty function. The ICO constraint helps obtain a shape that doesn't to require support by avoiding problematic overhang angles.

The release profiled for the three designs 730, 732, 734, are superimposed on the release curves 720, 722 that were used as design constraints. The simulated release curve 740 is for the designs with the minimal fabricability constraint. The simulated release curve 742 for the extruded design constraint 732 is also shown. The simulated release curve 744 for the ICO design constraint 734 is also shown. It can be seen that in most cases the release curves 740, 742, 744 match the cumulative release curve 720 or the instantaneous release curve 722 particularly well.

Design constraint 706, is of particular interest. It has a zero-order release curve (for the instantaneous release curve). It can be seen that the instantaneous release curve 722 for design 706, has a constant release which then rapidly drops to zero. This may be particularly useful in maintaining a constant release of a material. This may be useful for chemical reactions as well as providing highly controlled release of a pharmaceutical substance.

Below, an example of how to calculate a simulated release curve of a trial density function is described. This is done in the context of a gradient-based solution of the optimization problem. The calculation of the simulated release curve is generally applicable to other optimization schemes.

The solution of the optimization problem using a gradient-based optimization is enabled by constructing a differentiable forward model of the release process to provide a simulated release curve for a given object geometry Ω⊂R3.

A simplified geometric model for simulating the release of an object into a stirred solvent may be constructed. The fundamental underlying assumption of our model is that the entire exposed boundary of the object dissolves at the same rate. This rate will depend on the current concentration level in the solvent, but to simplify the exposition, we further suppose that the rate is constant. Concentration-dependent rates can also be handled by a nonlinear remapping of the simulation time, however this is not considered in the present example.

By defining the simulation time unit to be the length of time required to dissolve through a thickness of 1 mm, the time at which a given point x within a fully solid design Ω dissolves is the point's distance u(x) to the boundary ∂Ω of the trial density field.

Accordingly, the cumulative release at simulation time r can be computed directly from the distance field u: Ω→R⁺:

$$\begin{matrix}C ( τ ) = {\int }_{Ω} {ρ}_{0} \{ u < τ \} dx = {\int }_{Ω} {ρ}_{0} H ( τ - u ) dx , & H ( t ) := \{ \begin{matrix}1 & t > 0 \\ 0 & t \leq 0\end{matrix}\end{matrix} ,$$

where ρ₀ is the mass density of the fabrication material, and H is the Heaviside step function. The distance field u can be computed in a number of ways (e.g., fast marching or the Geodesics in Heat approach) and can be characterized as a viscosity solution of the unit-speed Eikonal equation:

$$\begin{matrix}‖ ∇ u ‖ = 1 & in & Ω \\ u = 0 & on & \partial Ω .\end{matrix}$$

The formula for C(τ) just presented has a few limitations. It can simulate only fully solid objects without holes, and it is not clear how to incorporate it in a topology optimization. To address both limitations, we generalize our simulation model by adopting a density-based object representation. The use of a trial density field overcomes these limitations.

Denoting the rectangular design domain (e.g., a three-dimensional printer's build volume) by [symbol] ⊂R3, the object is represented by a design density function ρ: [symbol] →[0, 1] (distinct from the physical material density ρ₀, which we assume in the following to be ρ₀=1 without a loss of generality). For instance, to represent Ω⊆ [symbol] , now possibly with holes, we take ρ to be the indicator function χ_{Ω}, which assumes value 1 inside Ω and 0 outside.

An insight for simulating an object of this type is to interpret low density values as material that dissolves at extremely high speed and solve a variable-speed Eikonal equation on [symbol] :

$$\begin{matrix}‖ ∇ u ‖ = 1 & in & 𝒟 \\ u = 0 & on & \partial 𝒟 .\end{matrix} \qquad ( 1 )$$

where n: [symbol] →(0, ∞) is the reciprocal dissolution speed field sometimes called the “slowness field” (speed is 1/n). This formulation automatically handles structures like interior holes and nested shells, where the inner shell starts dissolving immediately after the outer shell fully dissolves. One may use use an interpolation law to define n in terms of the design density:

$$n = ρ + ( 1 - ρ ) {n}_{min} \qquad ( 2 )$$

where nₘᵢₙ is a small value used to avoid numerical issues in the Eikonal equation solver associated with extreme speeds (nₘᵢₙ=10⁻⁶ is one value that works well). This simplest choice of a linear interpolation law is natural since one would expect, e.g., a half-occupied voxel to dissolve in half the time. One may penalize final designs (for example in the manufacturing penalty function) to be nearly binary, the precise choice of interpolation law only influences intermediate designs evaluated during the optimization.

After solving for the “dissolution time” field u, one may evaluate the cumulative release curve by integrating over the full design domain:

$$C ( τ ) = {\int }_{𝒟} ρ H ( τ - u ) dx . \qquad ( 3 )$$

Given a target release curve C* (τ), we formulate the inverse design problem as the optimization:

$${min}_{0 \leq \dot{ρ} \leq 1} {J}_{fit} ( ρ ) + {w}_{b} {J}_{binary} ( ρ ) + {e}_{f} {J}_{fab} ( ρ ) \qquad ( 4 )$$

$${J}_{fit} := \frac{1}{T} {\int }_{0}^{T} {( C ( τ ) - {C}^{*} ( τ ) )}^{2} d τ$$

$${J}_{binary} := {\int }_{𝒟} ρ ( 1 - ρ ) dx$$

$$ρ = project ( smooth ( \hat{ρ} , r ) , β ) ,$$

where smooth(⋅, r) is a standard smoothing filter with a kernel that linearly decays to 0 over the smoothing radius r, and project is the threshold projection filter:

$$project ( \tilde{ρ} , β ) := \frac{tanh ( β / 2 ) - tanh ( β ( \breve{ρ} - 1 / 2 ) )}{2 tanh ( β / 2 )} .$$

Together, these filters avoid nonphysical mesh-dependency issues like checkerboard patterns while retaining a binary design. J_{binary} is a standard term penalizing non-binary designs, and J_{fab} may be a term promoting robustly printable designs. The total simulation duration T is determined by the maximum possible dissolution time for a fully solid design; for a cube-shaped [symbol] , this is half the cube width.

The solution of these equations may be done by discretizing the design domain [symbol] with a regular grid of N cube voxels having edge length Δx. The density ρ is discretized as a piecewise constant field, meaning we define one design variable per voxel. When solving (1), one may pad the grid with a layer of zero density voxels on which the u=0 boundary condition is applied.

This may be used to ensure that parts of the object that intersect the domain boundary are not considered already dissolved at τ=0. One may solve Eq. 1 using an implementation of the fast marching method. The implementation used was modified to track the information necessary for performing the exact adjoint sensitivity analysis discussed in the next subsection. This method is based on a finite difference discretization of the Eikonal equation, and the code supports a second-order scheme for improved accuracy.

In some cases, the more complicated stencil selection rules involved in the second-order scheme degrade the differentiability of solution u (and thus the computed release curve). Because of this, the first-order scheme for inverse design. Denoting the dissolution time at voxel i by uᵢ and the neighbor of voxel i in the positive or negative direction along the d axis as N(i, ±d), the first-order scheme in three-dimensions solves the discrete equation

$$\begin{matrix}{\sum }_{d = 1}^{3} \frac{1}{{( Δ x )}^{2}} {max ( {𝓊}_{i} - {𝓊}_{𝒩 ( 𝒾 , 𝒹 )} , {𝓊}_{i} - {𝓊}_{𝒩 ( 𝒾 - 𝒹 )} , 0 )}^{2} = {𝓃}_{𝒾}^{2} & ∀ 𝒾 .\end{matrix} \qquad ( 5 )$$

Note that the max operation automatically selects the correct upwind stencil for each dimension. The dissolution time uᵢ determined by Eq. 5 indicates the time at which the voxel i has fully dissolved; this interpretation is apparent from studying a one-dimensional version of the discretization. It would be both nonphysical and yield a nondifferentiable model to assume that the entire voxel dissolves instantaneously at time uᵢ. Instead, one may assume that the voxel dissolves at a constant rate over the time interval (uᵢ−nᵢΔx, uᵢ). In other words, voxel i contributes to the release curve a scaled linear ramp function:

$${C}_{i} ( τ ) := ρ {i ( Δ x )}^{3} clamp ( \frac{τ - {𝓊}_{i}}{{𝓃}_{i} Δ x} + 1 , 0 , 1 ) . \qquad ( 6 )$$

Summing these contributions over the voxels obtains the following discretization of (3):

$$\tilde{C} ( τ ) := {\sum }_{i = 1}^{N} {\tilde{C}}_{i} ( τ ) = {\sum }_{i = 1}^{N} {ρ}_{i} \tilde{H} ( \frac{τ - {𝓊}_{i}}{{𝓃}_{i} Δ x} ) {( Δ x )}^{3} , \qquad ( 7 )$$

where \tilde{H}(τ):=clamp(τ+1, 0, 1) is a smeared version of the Heaviside function.

One may discretize the time integral in J_{fit} by picking a time step size Δτ and computing 1/TΣᵢ (\tilde{C}(iΔτ)−C*(iΔτ))2Δτ. One may use Δτ=T/1000 for some examples. One notes that a literal implementation of Eq. 7, summing up the full vector of concentration samples \tilde{C}(iΔτ), has computational complexity O(N T/Δτ), which becomes a bottleneck when high temporal resolution is requested. This can be accelerated to O(N+T/Δτ) by exploiting the fact that the second derivative of ramp function Eq. 6 is highly sparse, consisting of just two delta functions; therefore, we can accumulate the second finite difference of the discrete release curve in O(N) and then integrate it twice (with cumulative sums) to obtain \tilde{C}(iΔτ).

One may solve the discrete version of Eq. 4 using Knitro's L-BFGS implementation (an interior-point limited-memory Broyden-Fletcher-Goldfarb-Shanno (BFGS) algorithm). This algorithm may use the gradients of the design objectives J_{fit}, J_{binary}, J_{fab} with respect to the filtered density variables ρ which are then back-propagated through the density filter chain to obtain the gradients with respect to the design variables \hat{ρ}.

$${J}_{binary} = {\sum }_{i = 1}^{N} {ρ}_{i} ( 1 - {ρ}_{i} ) {( Δ x )}^{3}$$

can be directly differentiated, and J_{fab} may be defined and differentiated.

Differentiating J_{fit} is more challenging since it may use derivatives of the above-described forward model; one may obtain these efficiently with exact sensitivity analysis using the adjoint method.

Note that in the described inverse design formulation is not limited to the particular design representation and numerical optimization algorithms chosen by the described implementation. For instance, the topology optimization problem Eq. 4 can be re-cast in the level set framework wherein the geometry of the solid phase is represented by an implicit function and at, each iteration, is rasterized to the fast marching grid for discrete objective and gradient evaluation. Any largescale gradient-based optimization algorithm supporting bound constraints can be used in place of the L-BFGS interior point solver used for our results; the Method of Moving Asymptotes (MMA) is one such popular algorithm known to work well for general topology optimization problems with millions of variables.

Furthermore, even derivative-free optimization methods can be used, at the risk of lower performance and lower scalability to the number of density variables; genetic algorithms (GAs) and simulated annealing method are good candidates. Note that one of the penalty terms in J_{fab} aims to enforce near binary solutions to our optimization problem (i.e., nearly a pure solid-void design) in order to minimize the optimality sacrificed when the design is projected to a binary solution (either a simple thresholding or by contouring the density field and re-rasterizing it). An alternative is to generate fabricable designs directly from the optimized, continuous density field using approaches depending on the fabrication technology.

For example, stereolithography (SLA) three-dimensional printing or digital light processing (DLP) three-dimensional printing are known for their capabilities to create objects with intricate, volumetric microstructures (also known as foams) at the scale of tens of microns. For preparing such a design, the optimized, continuous density field obtained using our method can be converted into an object with such volumetric microstructures using a variety of methods, such as relying on procedural, aperiodic microstructures whose parameters can vary spatially across the whole design's geometry. By picking a specific one of these parametric families of microstructure geometries a mapping from material densities ρ to foam geometries can be established, and simulations or empirical measurements of these geometries can be used to develop a data driven interpolation law to replace Eq. 2.

The above-described framework extends in various ways to target fabrication technologies supporting the combination of multiple materials in a single part, such as inkjet or powder-bed inkjet three-dimensional printers. The simplest extension would be to a two-material composite wherein the void phase is replaced with a second material with finite density and dissolution speed. In this case, p denotes the volume fraction of the primary, release-controlled material in the mixture. Again, only the interpolation law Eq. 2 may be adapted based on the dissolution behavior of the resulting mixture. If ultimately a binary two-material design may be produced, the interpolation law can be a simple averaging; if the fabrication process supports mixing the materials at a fine scale (e.g., by dithering using a halftoning algorithm such as error-diffusion or iterative halftoning), then a data-driven interpolation law can be developed for the mixtures and a non-binary design can be produced. For composites of M≥3 materials (where one material might be void), two changes are needed. First, the interpolation law again may be developed (in the simplest case, linear interpolation can be used, taking the M volume fractions as weights). Second, the set of variables may be expanded to describe the multi-material design. One approach is to employ M−1 separate density (volume fraction) scalar fields as variables, where the volume fraction of the M^{th} material is obtained by subtracting the sum of the M−1 scalar fields from 1. The bound constraints in Eq. 4 would be applied to all M volume fraction fields.

If fine scale mixing of the materials is not supported by the targeted fabrication process and an “integer design” is required, the binary term of J_{fab} can be generalized promote designs in which exactly one of the volume fraction fields is nonzero at any point. For each additional material of the composite whose release is to be controlled, an additional term identical to Eq. 7 may be added to J_{fit} (with the respective material's volume fraction in place of ρ).

The above method also can be extended to support material- and process-related constraints for different fabrication systems. For instance, additional objective terms in J_{fab} can be developed to penalize the formation of narrow channels, which were shown to be problematic for materials that swell substantially during dissolution. Alternatively, morphology-based filters can be incorporated in the density filter pipeline to establish minimum feature length scales for these channel regions. Topology optimization techniques for enforcing the design rules of subtractive manufacturing and injection molding, both active research topics, can be incorporated into examples to obtain designs amenable to these technologies.

### LIST OF REFERENCE NUMERALS

100 manufacturing system
102 computer
104 computational system
106 hardware interface
108 user interface
110 memory
112 machine executable instructions
114 release profile
116 similarity cost function
118 simulated release curve
120 trial density field
122 optimization problem
124 manufacturing penalty function
126 optimized density field
128 post processing algorithm
130 three-dimensional design
200 receive a release profile for an object to be constructed of one or more materials
202 construct a similarity cost function dependent on a difference between a simulated release curve of a trial density field and the release profile for the object
204 construct an optimization problem comprising the similarity cost function and a manufacturing penalty function, wherein the manufacturing penalty function comprises one or more manufacturing constraints for a fabrication machine
206 construct an optimized density field by numerically solving the optimization problem to provide the trial density field that minimizes the optimization problem
208 apply a post processing algorithm to the optimized density field
210 provide a three-dimensional design of the object using the optimized density field that minimizes the optimization problem
300 manufacturing system
302 fabrication machine
304 object or tool
306 fabrication machine control commands
400 generate fabrication machine control commands using the three-dimensional design
402 fabrication machine to execute the fabrication machine commands
500 dissolution time field
502 voxel
504 voxel
506 voxel
600 graph of voxel release profiles
602 graph full object release profile
604 analytically calculated release curve
700 design constraint
702 design constraint
704 design constraint
706 design constraint
708 design constraint
710 design constraint
712 design constraint
720 cumulative release curve
722 instantaneous release curve
730 design using minimal fabricability design constraint
732 design using extruded design constraint
734 design using incremental compliance design constraint
740 simulated release curve for minimal fabricability design constraint
742 simulated release curve for extruded design constraint
744 simulated release curve for incremental compliance design constraint

1. A manufacturing system comprising:
a memory storing machine executable instructions; and
a computational system, wherein execution of the machine executable instructions causes the computational system to:
receive a release profile for an object to be constructed of one or more materials, wherein the release profile is descriptive of a dissolution of the one or more materials in a predetermined solvent;
construct a similarity cost function dependent on a difference between a simulated release curve of a trial density field and the release profile for the object;
construct an optimization problem comprising the similarity cost function and a manufacturing penalty function, wherein the manufacturing penalty function comprises one or more manufacturing constraints for a fabrication machine;
construct an optimized density field by numerically solving the optimization problem to provide the trial density field that minimizes the optimization problem; and
provide a three-dimensional design of the object using the optimized density field that minimizes the optimization problem.

2. The manufacturing system of claim 1, wherein a density of each of the one or more materials varies spatially.

3. The manufacturing system of claim 1, wherein the simulated release curve of the trial density field is formulated as an integral of a pointwise release curve of the trial density field.

4. The manufacturing system of claim 3, wherein the pointwise release curve of the trial density field is determined for each point within the trial density field by determining a distance from each point to a closest boundary of the trial density field and assuming a constant dissolution rate of each of the one or more materials in the predetermined solvent.

5. The manufacturing system of claim 4, wherein the distance of each point within the trial density field to the closest boundary of the trial density field is determined using a fast marching algorithm or a geodesics in heat algorithm.

6. The manufacturing system of claim 1, wherein the trial density field is discretized into voxels.

7. The manufacturing system of claim 6, wherein voxels contribute to the simulated release curve using a scaled linear ramp function.

8. The manufacturing system of claim 1, wherein the optimization problem comprises a density projection filter.

9. The manufacturing system of claim 1, wherein the optimization problem comprises a smoothing filter.

10. The manufacturing system of claim 9, wherein the smoothing filter linearly decays over a predetermined smoothing radius.

11. The manufacturing system of claim 1, wherein the optimization problem is formulated using a two-dimensional blueprint density field.

12. The manufacturing system of claim 11, wherein the computational system is configured to transform the two-dimensional blueprint density field into an extruded three-dimensional density field to provide the trial density field.

13. The manufacturing system of claim 1, wherein the optimization problem is solved using a gradient based optimization algorithm.

14. The manufacturing system of claim 13, wherein the gradient based optimization algorithm is an Interior-Point Limited-memory Broyden-Fletcher-Goldfarb-Shanno algorithm.

15. The manufacturing system of claim 1, wherein the optimization problem is solved using any one of the following: a Method of Moving Asymptotes algorithm, a genetic algorithm, and a simulated annealing method algorithm.

16. (canceled)

17. (canceled)

18. The manufacturing system of claim 1, wherein providing the three-dimensional design of the object using the optimized density field that minimizes the optimization problem comprises applying a post processing algorithm to the optimized density field.

19. The manufacturing system of claim 16, wherein the post processing algorithm is any one of the following: a halftoning algorithm, a microstructure approximation algorithm, and a surface contouring algorithm.

20. The manufacturing system of claim 1, wherein the release profile is a 0-order release profile.

21. The manufacturing system of claim 1, wherein the one or more materials is any one of the following: a single material, multiple materials, and a foam.

22. (canceled)

23. (canceled)

24. The manufacturing system of claim 19, wherein the foam is designed using holes, voids, or a porous microstructure.

25. The manufacturing system of claim 1, wherein at least one of the one or more materials comprises any one of the following: a pharmaceutical, a catalyst, an enzyme, a flavoring, a perfume, a deodorant, a pigment, an antibacterial agent, bioink, collagen, and a hydrogel.

26.-34. (canceled)

35. A computer program comprising machine executable instructions for execution by a computational system, wherein execution of the machine executable instructions causes the computational system to:
receive a release profile for an object to be constructed of one or more materials, wherein the release profile is descriptive of a dissolution of the one or more materials in a predetermined solvent;
construct a similarity cost function dependent on a difference between a simulated release curve of a trial density field and the release profile for the object;
construct an optimization problem comprising the similarity cost function and a manufacturing penalty function, wherein the manufacturing penalty function comprises one or more manufacturing constraints for a fabrication machine;
construct an optimized density field by numerically solving the optimization problem to provide the trial density field that minimizes the optimization problem; and
provide a three-dimensional design of the object using the optimized density field that minimizes the optimization problem.

36. A method, wherein the method comprises:
receiving a release profile for an object to be constructed of one or more materials, wherein the release profile is descriptive of a dissolution of the one or more materials in a predetermined solvent;
constructing a similarity cost function dependent on a difference between a simulated release curve of a trial density field and the release profile for the object;
constructing an optimization problem comprising the similarity cost function and a manufacturing penalty function, wherein the manufacturing penalty function comprises one or more manufacturing constraints for a fabrication machine;
constructing an optimized density field by numerically solving the optimization problem to provide the trial density field that minimizes the optimization problem; and
providing a three-dimensional design of the object using the optimized density field that minimizes the optimization problem.

37. (canceled)

38. The method of claim 23, wherein the wherein the release profile is a 0-order release curve.

39. (canceled)

40. An object manufactured according to the method of claim 23.
